(19) United States
(12) Plant Patent Application Publication
(10) Pub. No.: US 2005/0216998 P1
(43) Pub. Date: Sep. 29, 2005

(54) PETUNIA PLANT NAMED 'SUNCOPAHO'

(50) PETUNIA HYBRIDA; PETUNIA

(76) Inventors: Takuro Ishihara, Higashikurume (JP); Kazunari Iwaki, Omihachiman (JP); Kiyoshi Miyazaki, Hikone (JP); Shinya Miyano, Katori-gun (JP)

Correspondence Address: Christie, Parker & Hale, LLP;Suite 440, 350 West Colorado Boulevard, P.O. Box 7068, Pasadena, CA, 91109-7068, US

(21) Appl. No.: 10/810,495

(22) Filed: Mar. 25, 2004

(51) Int. Cl. A01H 5/00

(52) U.S. Cl. PLT/356

(57) ABSTRACT

Disclosed herein is a new and distinct variety of Petunia plant having a decumbent habit and short stems. The Petunia plant has abundant branching, and a great profusion of blooms, the whole plant remaining in blooin for a considerable period of time. The flowers are single and small, the petals having yellowish white color. The inside color of the corolla throat is light yellow green and the outside of the corolla tube is light yellow green. The plant exhibits high resistance to heat, cold and disease.

### BOTANICAL/COMMERCIAL CLASSIFICATION

Petunia hybrida/Petunia Plant

### VARIETAL DENOMINATION

cv. ‘Suncopaho’

### BACKGROUND OF THE VARIETY

The present invention relates to a new and distinct variety of Petunia plant, which originated from the crossing of a Petunia hybrid variety called ‘9Pt-27a’ as the female parent and ‘P59d-C10’ as the male parent.

The Petunia is a very popular plant used for flower bedding and potting in the summer season. There are only a few Petunia varieties that do not have an upright growth habit and that have a high resistance to rain, heat, and diseases. Petunias of the ‘Revolution’ series include ‘Revolution Purple pink’ (U.S. Plant Pat. No. 6,915), ‘Revolution Brilliant pink’ (U.S. Plant Pat. No. 6,914), ‘Revolution Brilliantpink-Mini’ (U.S. Plant Pat. No. 6,899), and ‘Revolution Blue vein’ (U.S. Plant Pat. No. 9,322). These are decumbent type plants having long stems, a lower plant height, abundant branching, and a high resistance to heat, rain and disease. However, there are only a few Petunia varieties having a decumbent and compact plant shape, a great profusion of small size flowers, white petals and a high resistance to rain, heat, and disease. Accordingly, this invention was aimed at obtaining a new Petunia variety having white petals, together with the above features.

The female parent ‘9Pt-27a’ (unpatented) used in the crossing of ‘Suncopaho’ is a strain of our breeding lines, having a spreading growth habit with many branches. It has medium size single flowers, the petals having a yellowish white color.

The male parent ‘P59d-C10’ (unpatented) used in the crossing of ‘Suncopaho’ is a strain of our breeding lines, having a decumbent growth habit with many branches. It has small single flowers, the petals having a yellowish white color.

In July 2000, crossing of 19Pt-27a, as the female parent and ‘P59d-C10’ as the pollen parent was conducted at Yokaichi-shi, Shiga-ken, Japan. In April 2001. 80 seedlings were obtained from that crossing. These seedlings were grown in pots in glasshouses and were evaluated. One seedling was selected in view of its growth habit, flower size and color in September 2001. That seedling was propagated by cutting and a trial was carried out by flower potting and bedding from April to September 2002 at Yokaicbi-shi, Shiga-ken, Japan. The botanical characteristics of that plant were then examined. using similar varieties Revolution white, (U.S. Plant Pat. No. 8,768) and ‘Sunchiffon’ (U.S. Plant Pat. No. 15,024) for comparison. As a result, it was concluded that this Petunia plant is distinguishable from any other variety, whose existence is known to us, and is uniform and stable in its characteristics. The new variety of Petunia plant was named ‘Suncopaho’.

In the following description, the color-coding is in accordance with the Horticultural Colour Chart of The Royal Horticultural Society, London, England (R.H.S. Colour Chart).

### SUMMARY OF THE VARIETY

This new variety is unlike any Petunia commercially available as evidenced by the following unique combinations of characteristics.

1. Rather compact and decumbent growth habit with short stems.

2. Having abundant branching and a great profusion of blooms.

3. The flowers are single and small. The petal color is yellowish white (near R.H.S. 158D).

4. The plant has a high resistance to cold, heat and disease.

The new variety ‘Suncopaho’ differs from the similar variety ‘Revolution White’ in the following points.

1. The spreading area of ‘Suncopaho’ is smaller than that of ‘Revolution White’.

2. The stem of ‘Suncopaho’ is shorter and thinner than that of ‘Revolution White’.

3. The internode length of ‘Suncopaho’ is shorter than that of ‘Revolution White’.

4. The leaf of ‘Suncopaho’ is smaller and thinner than that of ‘Revolution White’.

5. The flower size of ‘Suncopaho’ is smaller than that of ‘Revolution White’.

6. The shape of petal of ‘Suncopaho’ is very broadly obovate, while that of ‘Revolution white’ is acute.

The new variety ‘Suncopaho’ differs from the similar variety ‘Sunchiffon’ in the following points.

1. The spreading area of ‘Suncopaho’ is smaller than that of ‘Sunchiffon’.

2. The stem of ‘Suncopaho’ is shorter than that of ‘Sunchiffon’.

3. The leaf of ‘Suncopaho’ is smaller and thinner than that of ‘Sunchiffon’.

4. The petal color of ‘Suncopaho’ is yellowish white (near R.H.S. 158D), while that of ‘Sunchiffon’ is moderate purplish pink (near R.H.S. 68C).

5. The apex shape of petal of ‘Suncopaho’ is rounded, while that of ‘Sunchiffon’ is obtuse.

6. The petal lobation of ‘Suncopaho’ is deeper than that of ‘Sunchiffon’.

The new variety of Petunia plant ‘Suncopaho’ was asexually reproduced by the use of cuttings at Yokaichi-shi, Shiga-ken, Japan, and the homogeneity and stability thereof were confirmed. The instant plant retains its distinctive characteristics and reproduces true to type in successive generations.

### BRIEF DESCRIPTION OF THE PHOTOGRAPH

The depicted plants had been reproduced by the use of cuttings and were photographed during July 2003 while cultivating under the trial field in 15 cm pots at an age of approximately 6 months at Yokaichi-shi, Shiga-ken, Japan.

FIG. 1 is a photograph of a typical plant of the new variety of Petunia plant ‘Suncopaho’ while growing in a pot.

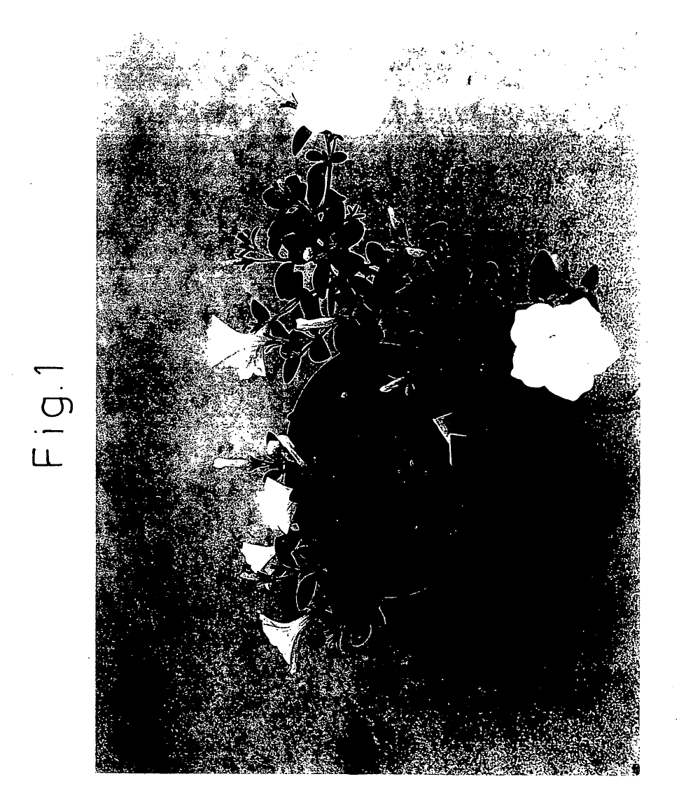

FIG. 2 is a photograph of a close view of flowers and leaves of the new variety of Petunia plant ‘Suncopaho’.

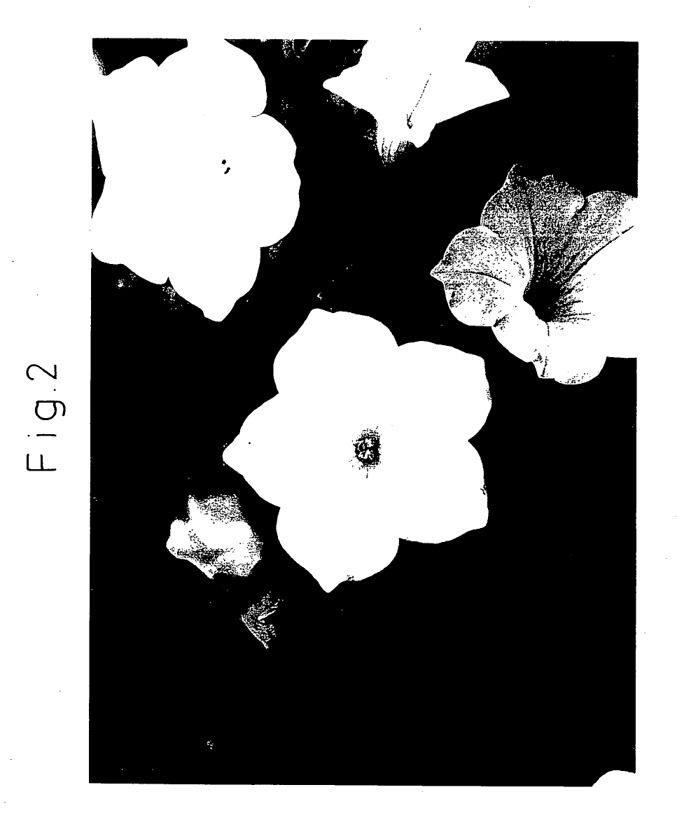

### DESCRIPTION OF THE VARIETY

The botanical characteristics of the new and distinct variety of Petunia plant named ‘Suncopaho’ are as follows when observed during July at Yokaichi-shi, Shiga-ken, Japan, at an age of approximately 6 months.
Plant:
   Growth habit.—Decumbent.
   Plant height.—Approximately 11.3 cm.
   Spreading area of plant.—Approximately 25.0 cm.
   Blooming period.—Early April to late October in the southern Kanto area, Japan. The plant shape does not change throughout this period. A typical flower commonly lasts approximately 5 days on the plant when experiencing a temperature of approximately 20° C.
Stem:
   Length.—Approximately 2.0 cm.
   Thickness.—Approximately 1.4 mm.
   Pubescence.—Sparse.
   Branching.—Abundant.
   Internode length.—Approximately 1.5 cm.
   Color.—Near R.H.S. 146B (moderate olive green).
Leaf:
   Whole shape.—Elliptic with entire margin. The apex shape is acute, and the base shape is attenuate.
   Length.—Approximately 3.2 cm.
   Width.—Approximately 1.9 cm.
   Color.—Upper side color is near R.H.S. 147A (moderate olive green). Lower side color is near R.H.S. 146A (moderate olive green).
   Thickness.—Approximately 0.1 mm.
   Pubescence.—Sparse.
Buds:
   Shape.—Cylindrical.
   Length.—Approximately 3.5 cm.
   Diameter.—Approximately 6.0 mm.
   Color.—Near R.H.S. 149C with venation near N144D.
Flower:
   Petals: Width — Approximately 2.5 cm. Length from throat — Approximately 2.0 cm. Shape — Very broadly obovate. Margin — Entire. Texture — Smooth. Color — Lower surface — near R.H.S. 158D with venation near R.H.S. N144D.
   Facing direction.—Slanted upward.
   Type.—Single.
   Shape.—Funnel-shape, with five-fissures.
   Shape of petal tip.—Rounded.
   Lobation.—Medium.
   Waviness of petal.—Medium.
   Diameter.—Approximately 4.1 cm.
   Depth.—Approximately 4.0 cm.
   Tube length.—Approximately 2.3 cm.
   Throat diameter, distal end.—Approximately 1.0 cm.
   Tube diameter, proximal end.—Approximately 4.0 mm.
   Color.—Near R.H.S. 158D (yellowish white). Inside color of the corolla throat; Near R.H.S. 154C (light yellow green). Outside color of the corolla tube; Near R.H.S. 154C (light yellow green).
   Reproductive organs.—1 normal pistil and 5 normal stamens. Color of pistil is near R.H.S. 154C (light yellow green). Color of stamen is near R.H.S. 144D (light yellow green).
Peduncle:
   Length.—Approximately 7.0 mm.
   Diameter.—Approximately 1.0 mm.
   Color.—Near R.H.S. 144A.
   Surface.—Pubescent.
Sepals:
   Shape.—Narrow elliptic.
   Apex shape.—Rounded.
   Base.—Fused.
   Margin.—Entire.
   Surface.—Pubescent.
   Length.—Approximately 0.8 cm.
   Width.—Approximately 2.0 mm.
   Color.—Upper surface — near R.H.S. 146B, lower surface — near R.H.S. 146B.
   Calyx.—Narrow. 5 sepals fused at the base.
   Physiological and ecological characteristics.—High resistance to cold, heat and disease. Moderate resistance to rain and pests.

This new variety of Petunia plant is most suitable for flower bedding and potting, particularly in hanging pots or planters. Pinching of old blossoms will enhance the formation of new blossoms.

1. A new and distinct variety of Petunia plant named ‘Suncopaho’, substantially as herein illustrated and described.
